(19) United States
(12) Patent Application Publication
(10) Pub. No.: US 2025/0221894 A1
(43) Pub. Date: Jul. 10, 2025

(54) Delivery System

(71) Applicant: Bee Well Therapeutics LLC, Ormond Beach, FL (US)

(72) Inventor: Andrew Scott Bernett, Ormond Beach, FL (US)

(73) Assignee: Bee Well Therapeutics LLC, Ormond Beach, FL (US)

(21) Appl. No.: 19/093,552

(22) Filed: Mar. 28, 2025

Related U.S. Application Data
(63) Continuation-in-part of application No. 18/225,797, filed on Jul. 25, 2023.
(60) Provisional application No. 63/461,235, filed on Apr. 21, 2023.
(60) Provisional application No. 63/687,348, filed on Aug. 27, 2024.

(51) Int. Cl. A61K 8/02; A61K 8/92; A61K 8/9789; A61Q 19/00

(52) U.S. Cl. CPC A61K 8/027; A61K 8/0208; A61K 8/927; A61K 8/9789; A61Q 19/007; A61K 2800/5922; A61K 2800/87

(57) ABSTRACT

The present disclosure provides a delivery system comprising a fibrous substrate with continuous pores and a composition homogenously bonded to the pores. The fibrous substrate comprises at least a predetermined percentage of cotton and one or more materials selected from the group of consisting spandex, nylon, silver and antimicrobial mesh. The composition comprises a predetermined percentage of beeswax and a predetermined percentage of mineral oil. The predetermined percentage of beeswax is greater than the predetermined percentage of mineral oil.

### CROSS-REFERENCE TO RELATED APPLICATION

This application is a continuation-in-part of U.S. patent application Ser. No. 18/225,797, filed on Jul. 25, 2023, which claims the benefit of the filing date under 35 U.S.C. § 119(e) of Provisional Patent Application Ser. No. 63/461,235, filed Apr. 21, 2023. The application also claims the benefit of U.S. Provisional Application No. 63/687,348, filed Aug. 27, 2024. Each of the applications is hereby incorporated by reference in its entirety for all purposes.

### FIELD OF THE DISCLOSURE

The subject matter disclosed herein generally relates to delivery systems and more specifically to a delivery system that can be used with topicals and provides occlusion and permeability to the skin.

### BACKGROUND

Healthy skin contributes to an individual's appearance and may positively impact one's self-esteem. On the other hand, an unhealthy or diseased skin condition may affect one's life due to discomfort, and may be a health indicator for underlying health issues.

Currently, unhealthy skin conditions (including, but not limited to, dry skin, Atopic dermatitis, Eczema, Psoriasis, and Scleroderma) many people. Among all those skin conditions, dry skin is one of the most common issues, and may be caused by several reasons, such as frequent hand washing, use of hand sanitizers, and conditions of the environment and weather. Moreover, excessively dry skin may be uncomfortable and cause itching, peeling of skin, scaling or cracking.

Current treatments for extremely dry skin conditions include applying prescription topical ointments or lotions to the skin and cover with cotton or rubber materials to hold the ointment onto the dry skin and to prevent scratching while sleeping. However, the untreated cotton materials absorb a great amount of medication, leaving only a fraction of the medication being applied to the skin. Rubber has better occlusion, but consequently creates a maceration problem if left on the skin for too long. For other skin conditions (such as Atopic dermatitis, and white cotton), plastic materials and wet wraps are typically utilized, which may cause additional side effects and may not be ideally effective.

Therefore, it is desirable to provide a delivery system that can be used with a variety of topicals and provides occlusion and permeability to the skin.

### SUMMARY OF THE DISCLOSURE

The following is a concise summary of the disclosure presented herein with the primary aim of providing a preliminary understanding of certain aspects of the disclosure. It should be noted, however, that this summary is not intended to serve as a comprehensive overview of the disclosure, nor does it seek to identify or describe any critical or significant elements of the disclosure or the boundaries of its scope. Its sole purpose is to provide a rudimentary understanding of the disclosure's concepts and features, which will be expounded upon in greater detail in the ensuing sections.

The present disclosure is generally directed towards a delivery system, and specifically directed towards a delivery system bonded with a beeswax composition that may be applied in combination with topicals, lotions, gels, skin care products, moisturizers, prescriptions, or active ingredients. The delivery system may be applied to various dermal regions and body parts to facilitate moisturizing skin. Advantageously, the breathability and the occlusion of the delivery system provide an optimal environment for skin absorption.

A non-limiting exemplary embodiment (the “exemplary embodiment”) of the present disclosure provides a delivery system and is disclosed herein. The exemplary embodiment comprises a fibrous substrate having continuous pores and a composition bonded to the pores. In the exemplary embodiment, the fibrous substrate comprises at least a predetermined percentage of cotton. It is preferable that the predetermined percentage of cotton is at least 50 percent with respect to total weight of the fibrous substrate. The composition comprises a predetermined percentage of beeswax and a predetermined percentage of mineral oil, and is homogeneously bonded (preferably thermally bonded) to the pores of the substrate. The predetermined percentage of beeswax is greater than the predetermined percentage of mineral oil. The predetermined percentage of beeswax is preferably 65 to 75 percent with respect to total weight of the composition. The predetermined percentage of mineral oil is preferably 25 to 35 percent with respect to total weight of the composition. The composition forms as the fibrous substrate was impregnated in a mixture, treated with isopropanol, and subsequently thermally cured. In some circumstances, the delivery system has an occlusion rate of less than 4000 g/m²·day as tested according to ASTM E96, and in other instances, less than 3000 g/m²·day.

In some embodiments, besides the predetermined percentage of cotton, the fibrous substrate may further comprise one or more materials selected from the group of consisting of polyester, spandex, nylon, silver and antimicrobial mesh. In some instances, the fibrous substrate comprises at least a predetermined percentage of cotton and a predetermined percentage of polyester. Preferably, the predetermined percentage of cotton is about 50 percent up to 100 percent, and the predetermined percentage of polyester is about less than 1 percent up to 50 percent by weight with respect to total weight of the fibrous substrate. In some circumstances, the predetermined percentage of cotton is about 70 percent, and the predetermined percentage of polyester is about 30 percent, in other instances, the predetermined percentage of cotton is about 75 percent, and the predetermined percentage of polyester is 25 percent. Yet in another embodiment, the predetermined percentage of cotton is about 50 percent, and the predetermined percentage of polyester is about 50 percent by weight with respect to total weight of the fibrous substrate.

In another aspect, the composition bonded to the delivery system comprises a predetermined percentage of beeswax, a predetermined percentage of mineral oil, and a predetermined percentage of cholesterol. Preferably, the predetermined percentage of beeswax is 70 percent, the predetermined percentage of mineral oil is 25 percent, and the predetermined percentage of cholesterol is 5 percent with respect to a total weight of the composition.

Yet in an alternative aspect, the fibrous substrate comprises at least two layers, such as an inner layer and an outer layer. Preferably, the inner layer comprises cotton and the outer layer comprises polyester.

In another aspect, a method for manufacturing the delivery system is disclosed herein. The method includes of providing a fibrous substrate including at least a predetermined percentage of cotton; heating a predetermined quantity of beeswax with a predetermined quantity of mineral oil over heat to a predetermined temperature to form a pliable mixture; saturating the fibrous substrate with the mixture until visual clues or auditory cues are provided to signal uniform impregnation; drying the fibrous substrate; applying isopropanol to the fibrous substrate; applying a detergent wash to the fibrous substrate; enclosing the fibrous substrate in a cloth sleeve to allow the sleeve to absorb the excessive mixture; and thermal curing the fibrous substrate by warm air and drying at a predetermined temperature such that the mixture forms a composition bonded to the fibrous substrate.

In some embodiments, disclosed herein is a mixture comprising a predetermined quantity of cholesterol, thereby forming a composition comprising beeswax, mineral oil and cholesterol.

Advantageously, when applying the delivery system to dry skin for at least a period of time, dry skin is relieved and moisturized. Further, having at least a predetermined percentage of cotton with the thermally bonded composition provides a suitable water vapor transmission rate, which further provides effective occlusion and breathability to the skin. The breathability is suitable for individuals having diabetes. With the combination of topicals, dermal conditions (such as eczema, psoriasis, dry skin, atopic dermatitis, scleroderma, and prurigo nodularis) may be relieved. Specially, it has been observed that individuals having scleroderma are benefited from the delivery system. Furthermore, notably in some implementations in podiatry, it has been effective for some individuals in softening callus by applying the delivery system with 40% urea cream. Furthermore, the delivery system is designed to have a specific weight of 160 grams to 250 grams per square meter.

The above features and advantages will become apparent from the following detailed description taken with the accompanying drawings.

### BRIEF DESCRIPTION OF THE DRAWINGS

FIG. 1 depicts a perspective view of an exemplary embodiment of a delivery system.

FIG. 2 depicts an enlarged view of a portion of the exemplary embodiment of the delivery system.

FIG. 3 depicts untreated fibrous substrate.

FIG. 4 depicts a perspective view of an alternative embodiment of the delivery system.

FIG. 5 depicts a cross-sectional view taken along line 5-5 in FIG. 4.

FIG. 6 is the diagram showing a method of manufacturing the delivery system.

### NUMBER REFERENCES

1—Exemplary Embodiment of the delivery system
10—Fibrous Substrate
11—Cotton
12—Polyester
13—Pores
15—Composition
2—Alternative Embodiment of the delivery system
21—Inner Layer
22—Outer Layer
25—Composition

### DETAILED DESCRIPTION OF THE DISCLOSURE

The following detailed description and accompanying drawings provide a comprehensive disclosure of exemplary embodiments for the purpose of facilitating one of ordinary skill in the relevant art to make and use the disclosure. Therefore, the detailed description and illustration of the one or more exemplary embodiments presented herein are purely exemplary in nature and are not intended to limit the scope of the disclosure or its protection in any manner. It is further noted that the drawings may not be to scale, and in some cases, certain details may be omitted which are not necessary for an understanding of the present disclosure, such as conventional details of fabrication and assembly.

As described herein, various compositional ranges do not exceed a hundred percent in total and unless indicated otherwise, the numerals of various compositional ranges presented herein are approximations.

As used in the following detailed description, the term “fibrous substrate” may refer, but is not limited to a patch, a bandage, a baby suit, a glove, a sock, a sleeve, or other garments having filaments and/or fibers having continuous void spacings or pores that can absorb liquids, semi-solid liquids or a mixture of liquids, semi-solid liquids.

As used in the following detailed description, the term “delivery system” may refer to a system that can be used individually or in combination with formulations for administrating across the skin. The formulations may include but are not limited to topicals, lotions, gels, skin care products, moisturizers, prescriptions, and active ingredients, and are applied to the skin by placing the delivery system to a dermal region for a predetermined period of time, such that the formulations may be applied to or delivered through the skin.

As used in the following detailed description, the term “untreated fibrous substrate” may refer to fibrous substrates with no composition bonded to the pores and has not gone through impregnation in a mixture comprising beeswax and mineral oil, isopropanol application, or thermal curing process. In some implements, the “untreated fibrous substrate” may undergo the process of cleaning or washing treatments.

As used in the following detailed description, the term “treated fibrous substrate” may refer to fibrous substrates that has gone through impregnation in a mixture comprising beeswax and mineral oil, isopropanol application, or thermal curing process, such that the mixture forms a composition comprising beeswax and mineral oil bonded to the fibrous substrate.

A non-limiting, exemplary embodiment (hereinafter as “the exemplary embodiment”) of a delivery system 1 is disclosed herein. The delivery system 1 may be utilized individually or in combination with formulations including but not limited to topicals, lotions, gels, skin care products, moisturizers, prescriptions, and active ingredients, to provide moisturization to the skin. The delivery system 1 provides both breathability and occlusion for skin absorption.

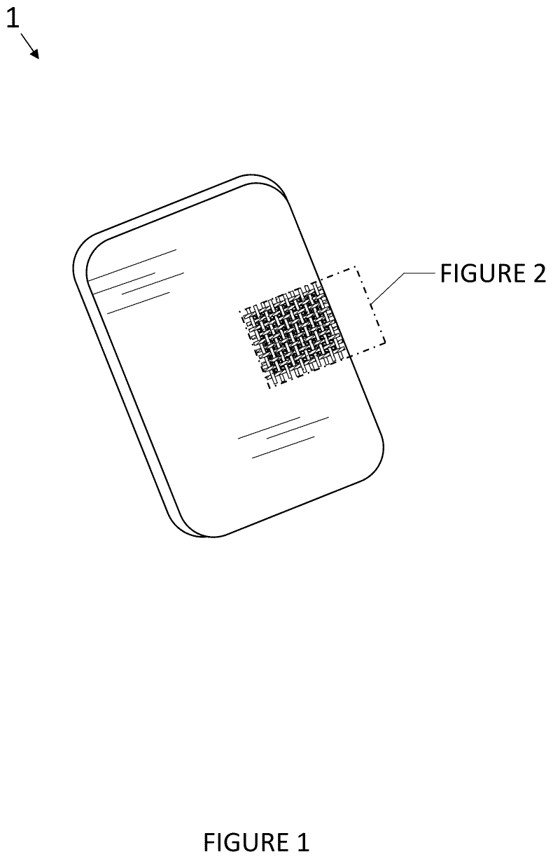

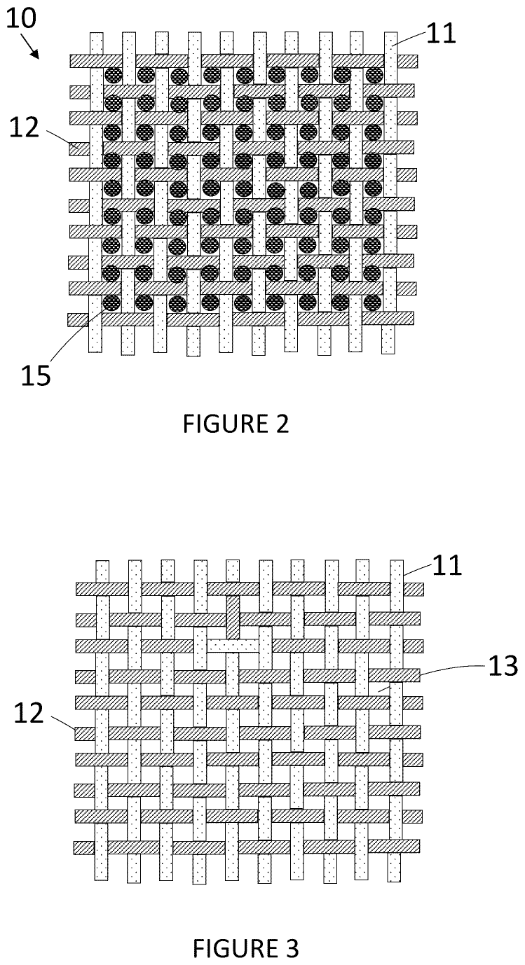

Accordingly, referring initially to FIGS. 1 2, and 3, a delivery system 1 is illustrated in accordance with the exemplary embodiment. FIG. 2 illustrates an enlarged view of the delivery system 1 of FIG. 1. The delivery system 1 comprises a fibrous substrate 10, which is comprised of at least a predetermined percentage of cotton 11. The predetermined percentage of cotton 11 is about at least 50 percent with respect to the total weight of the fibrous substrate 10. As illustrated in the exemplary embodiment, the fibrous substrate 10 further comprises a predetermined percentage of polyester 12. Preferably, the fibrous substrate 10 has a weight of 160 to 250 grams per square meter The fibrous substrate 10 further comprises continuous pores 13. The weight of the fibrous substrate 10 per square meter may vary according to different kinds of fibrous substrates 10. Heavier weight may be used in fibrous substrates 10 such as sleeves or gloves, while lighter weight should be used in fibrous substrates 10 such as baby suits to avoid babies from overheating.

A composition 15 is homogeneously bonded to the pores 13 (shown in FIG. 3) of the fibrous substrate 10. The composition 15 comprises a predetermined percentage of beeswax and a predetermined percentage of mineral oil, and the predetermined percentage of beeswax is greater than the predetermined percentage of mineral oil. The beeswax can be from any suitable beeswax supplier (such as, for example, Mary Taylor Naturals from Myers, Florida, USA). Preferably, the predetermined percentage of beeswax is preferably 65 to 75 percent with respect to total weight of the composition. The predetermined percentage of mineral oil is preferably 25 to 35 percent with respect to total weight of the composition. The composition 15 may further comprise at least one of Silicone Alcohol, Fatty Acids, Aliphatic Alcohols, Fatty Alcohols, Esters, Ketones, Dione, and Alkanes, such as Dimethylsilanediol, Capric Acid, Lauric Acid, 5-Methyl 1 Heptanol, Myricyl Palmitate, Myricyl Stearate, Myricyl Palmitate, Myricyl Cerotate, Myricyl Cerotate, Myricyl Behenate, Myricyl Arachidate, Myricyl, Benzophenone, Dione, and Hexatriacontane.

In some embodiments, the fibrous substrate 10 may comprise 100 percent cotton 11, and in other instances, the predetermined percentage of cotton 11 is about 50 to 100 percent, and the predetermined percentage of polyester 12 is about less than 1 percent to 50 percent by weight with respect to total weight of the fibrous substrate 10. For instance, the predetermined percentage of cotton 11 may be 50 to 75 percent by weight with respect to total weight of the fibrous substrate 10, and the predetermined polyester percentage of 25 to 30 percent by weight with respect to total weight of the fibrous substrate 10.

In other circumstances, the fibrous substrate 10 may further comprise one or more materials selected from the group of consisting spandex, nylon, silver and antimicrobial mesh. In some examples, the cotton 11 is preferably in a range of between 50 to 80 percent by weight of the fibrous substrate 10. In some instances, the polyester 12 may vary between 15 to 48 percent by weight of the fibrous substrate 10, and in other examples, spandex fibers (not shown) is preferably in a range of between 1 to 4 percent by weight of the fibrous substrate. Nylon may be up to 2 percent by weight of the fibrous substrate 10. Besides the foregoing combinations of materials for the exemplary embodiment and the respective alternative embodiments, the alternative embodiments may have the following ratios by weight percentage. In one example, the ratio may be 52 percent of cotton 11, to 47 percent of the polyester 12, to 1 between the cotton 11, the polyester 12 and the spandex fibers. In another example, the ratio may be 64 percent of cotton 11, 32 percent of polyester 12, and 3 percent nylon, and 1 percent spandex. In a further example, the ratio may be 65 percent of cotton 11, 30 percent of polyester 12, 4 percent of spandex and 1 percent of nylon. The addition of silver or antimicrobial mesh (not shown) can be added to the delivery system 1 for antibacterial purposes and to mitigate the possibility of infection. In some embodiments, the composition 15 further comprises a predetermined percentage of cholesterol.

It is anticipated that the cotton 11, the polyester 12 and other materials of the fibrous substrate 10 can be blended at a fiber stage, joined or interwoven by warp and weft at a yarn stage or alternative techniques for joining the distinct materials without departing from the spirit of the disclosure.

The following data including test conditions and test results demonstrates the occlusion and permeability of the disclosed delivery system 1 with different compositions. Standard Test methods for water vapor transmission of materials (ASTM E96) were followed and the desiccant method was utilized.

### Example 1

### Test Conditions

| Water Vapor | Temperature | Test Area |
|---|---|---|
| 50% relative humidity (RH) | 32.2° C. | 30 cm² |

### Test Results

Water Vapor Transmission Rate

| Sample | Specimen | g/(m² · day) |
|---|---|---|
| Untreated fibrous substrate | 1 | 1490 |
| with 100% cotton | 2 | 1460 |
| | 3 | 1460 |
| Treated fibrous substrate | 1 | 1330 |
| with 100% cotton | 2 | 1360 |
| | 3 | 1270 |

The purpose of the first test was to compare the Water Vapor Transmission Rate (WVTR) between untreated and treated fibrous substrates to understand the occlusion and permeability of untreated and treated fibrous substrates. Specimens 1-3 of the untreated fibrous substrate with 100% cotton are 30 cm² of fibrous substrate from an untreated baby suit with 100% cotton. The untreated baby suit has only undergone cleaning treatments, but has not gone through impregnation in a mixture, isopropanol application, or thermal curing process. On the contrary, specimens 1-3 of the treated fibrous substrate with 100% cotton are 30 cm² of fibrous substrate from a treated baby suit with 100% cotton. The treated baby suit was impregnated in a mixture comprising beeswax and mineral oil, and subsequently treated with isopropanol and thermally cured, such that a composition comprising beeswax and mineral oil is bonded to the fibrous substrate. Specifically, the aforementioned specimens are tested in a controlled atmosphere of 32.2° C. and 50% relative humidity, according to ASTM E96.

As indicated in the above results, the treated fibrous substrate has lower water vapor transmission rates, which provides better occlusion for skin absorption. As a result, the water vapor transmission rate reduces after the fibrous substrate was treated.

### Example 2

### Test Conditions

| Water Vapor | Temperature |
|---|---|
| 50% relative humidity (RH) | 32.2° C. |

### Test Results

Water Vapor Transmission Rate

| Sample | Specimen | g/(m² · day) |
|---|---|---|
| A: Treated fibrous substrate | 1 | 2400.7688 |
| with 50% cotton and 50% | 2 | 2700.8625 |
| polyester from gloves | | |
| B: Treated fibrous substrate | 1 | 566.0899 |
| with 70% cotton and 30% | 2 | 559.2695 |
| polyester from socks | | |
| C: Treated fibrous substrate | 1 | 43.1956 |
| with 70% cotton and 30% | | |
| polyester from a sleeve | | |

The purpose of the second test is to understand the occlusion level and permeability or breathability from various types of fibrous substrates. The above test utilizes treated fibrous substrate from gloves, socks and sleeves with distinctive percentages of cotton and polyester. Specimens 1 and 2 of sample A are from gloves that comprises treated fibrous substrate with 50% cotton and 50% polyester. Specimens 1 and 2 of sample B are from socks that comprises treated fibrous substrate with 70% cotton and 30%. Specimens 1 of sample C is from a sleeve with 70% cotton and 30% polyester. All specimens are tested in a controlled atmosphere of 32.2° C. and 50% relative humidity, according to ASTM E96.

As shown in the results above, each of the specimens has a water vapor transmission rate lower than 3000 g/(m²·day), which provides an optimal environment for skin absorption with both occlusion and breathability. Furthermore, a water vapor transmission rate around 1700-2300 g/(m²·day) may maintain optimal moisture for the proliferation and function of epidermal cells and fibroblasts, and facilitate wound healing.

The following data including test conditions and test results demonstrates the delivery system 1 also provides great compression to an individual's body parts. Furthermore, the test is done according to a modified BS 661210 standard for graduated compression hosiery, anti-embolism hosiery, and graduated support hosiery. Graduation of the hosiery is not tested.

### Test Conditions

| Water Vapor | Temperature |
|---|---|
| 65% ± 5% relative humidity (RH) | 21 ± 2° C. |

The following specimens in Test 1 are treated sleeves in size large, and two locations on the treated sleeves are tested.

### Test 1

| | First Location of | Second Location of |
|---|---|---|
| Specimens | the sleeve | the sleeve |
| Specimen 1 | 15.3 mmHg | 23.2 mmHg |
| Specimen 2 | 13.4 mmHg | 23.6 mmHg |
| Specimen 3 | 14.2 mmHg | 22.2 mmHg |
| Specimen 4 | 9.1 mmHg | 23.7 mmHg |
| Average Compression | 13.0 mmHg | 23.2 mmHg |

The following specimens in Test 2 are untreated sleeves in size large, and two locations on the treated sleeves are tested.

### Test 2

| | First Location of | Second Location of |
|---|---|---|
| Specimens | the sleeve | the sleeve |
| Specimen 1 | 6.7 mmHg | 15.7 mmHg |
| Specimen 2 | 6.6 mmHg | 13.2 mmHg |
| Average Compression | 6.7 mmHg | 14.5 mmHg |

As indicated in the above results, the treated sleeves provide a better compression than the untreated sleeves. Advantageously, the delivery system 1 provides may improve circulation, reduce swelling and be beneficial to diseases such as dermatitis, lymphedema, deep vein thrombosis (DVT), ulcers, edema and so forth.

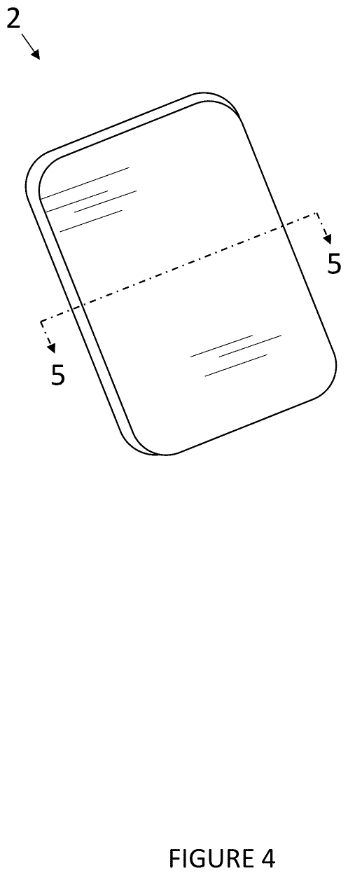

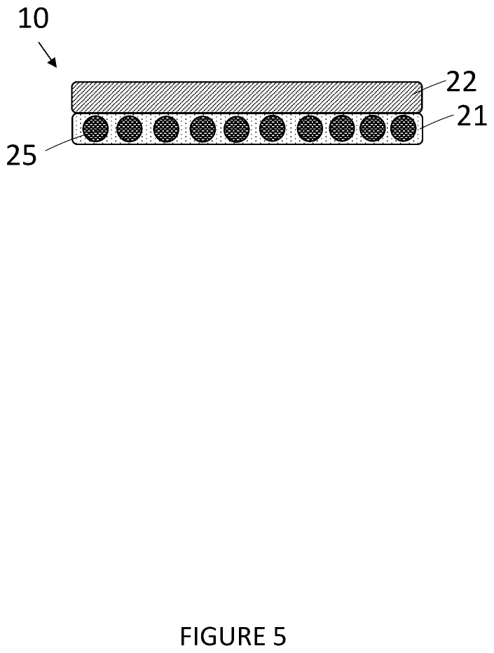

Referring to FIGS. 4 and 5, in an alternative embodiment, the delivery system 1 comprises at least two layers 21, 22. In some examples, the at least two layers 21, 22 include an inner layer 21 comprising cotton and an outer layer 22 comprising polyester. The distinct characteristics of cotton (i.e., higher hydrophilicity, greater porosity, and so forth) and polyester (i.e., higher hydrophobicity, smaller porosity) endow the two layers 21, 22 with distinct absorption of the mixture of beeswax and mineral oil. As such, due to the hydrophilicity of the inner layer 21 and hydrophobicity of the outer layer 22, when the glove 2 is impregnated with the mixture, the mixture has a tendency to substantially bind to the inner layer 21.

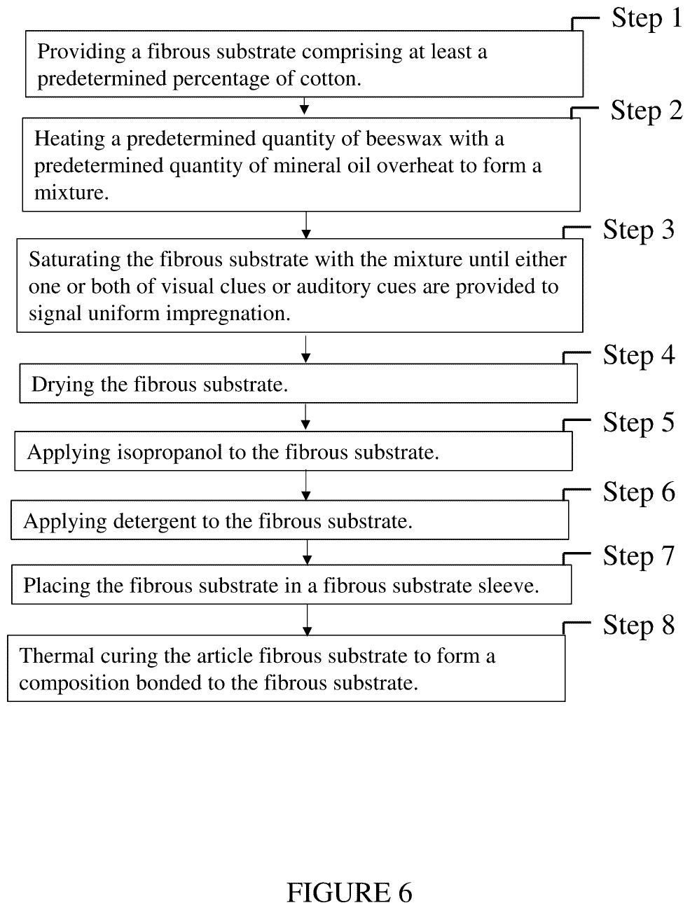

Turning to an exemplary method of manufacturing the delivery system 1, as shown in FIG. 6, the exemplary method is presented herein to further demonstrate how the composition forms and bonds to the fibrous substrate 10. It is anticipated that several steps may be sequentially interchangeable and equivalent application of one or more permutations of such sequentially interchangeable steps does not alter the spirit of the disclosure in any meaningful way.

In step 1 of the exemplary method, an untreated fibrous substrate 10, as shown in FIG. 3, comprised of at least a predetermined percentage of cotton 11 is provided for initiating the process. The untreated fibrous substrate 10 is then impregnated with a mixture of beeswax and mineral oil.

An optional pretreatment step of prewashing may be included if needed to remove contamination from the fibrous substrate 10. Examples of the prewashing include either by hand or by machine. The exemplary embodiment is prewashed, preferably by a washing machine, with a detergent to water ratio which may be approximately 110 liters of water to 60 to 80 milliliters of detergent under or below room temperature, such that the fibrous substrate 10 is not overheated to cause porosity of the fibrous substrate 10 to decrease. It is anticipated that bleach may also be added to the foregoing detergent and water. It is further anticipated that the method of prewashing and the foregoing mixtures of water, detergent, and possibly bleach, may vary.

In step 2, a mixture is formed by heating, preferably, beeswax in an amount of about 65 percent to about 75 percent by weight with mineral oil in an amount of about 25 percent to about 35 percent by weight at a predetermined temperature and mixed until well blended. More preferably, the beeswax of the mixture is in the amount of about 70 percent by weight and the mineral oil is in the amount of about 30 percent by weight. Examples of mixing the mixture may include, but not limited to, stirring, agitating, or whisking. The heating of the mixture may include heating over direct heat or indirect heat, and for example, may be heated by utilizing a double boiler. The predetermined temperature of the mixture should ideally be controlled lower than 120 degrees Celsius, preferably, 100 degrees Celsius to prevent the beeswax from burning or overheating. The choice of beeswax may include white beeswax or yellow beeswax. However, white beeswax that is filtered three times is preferred, because pollen and impurities from yellow beeswax are removed. The mineral oil adjusts the viscosity of the mixture and provides a soft but pliable texture. Moreover, mineral oil is food safe, uncommon to cause allergy reactions, and has low reactivity and a suitable boiling point.

In step 3, saturation of the untreated fibrous substrate 10 is performed. Examples of the saturation process may include soaking, dipping, physically kneading, implementation of a vacuum chamber, or any combination thereof. The untreated fibrous substrate 10 is saturated in the mixture 15 until visual clues or auditory cues are provided to signal uniform impregnation. In the exemplary embodiment, the saturation of the fibrous substrate 10 is performed within a vacuum chamber with an atmosphere of about 0.75. As the air is removed from the vacuum chamber, bubbles form in the mixture. Once the bubbles in the mixture have ceased to be visible or audible, the mixture is impregnated into the pores 13 of the fibrous substrate 10 evenly.

In step 4, the fibrous substrate 10 is dried, preferably airdried in ambient temperature, and then, in step 5, treated with isopropanol, preferably by hand and with isopropanol which is about 75 percent isopropanol. Afterwards, in step 6, a mild detergent, such as soap, is applied to the fibrous substrate 10 to remove the isopropanol of the fibrous substrate, thereby reducing the possibility of a reaction when applying the delivery system 1 to dry skin. Preferably, the detergent to water ratio is approximately 113 liters of water to 60 to 80 milliliters of detergent.

In step 7, a thermal curing process is performed to the fibrous substrate 10. The fibrous substrate 10 is placed in a cloth sleeve, preferably a cotton sleeve, and into a clothes dryer to absorb residuals. In step 8, the remaining mixture 15 is thermally cured at a temperature lower than 120 degrees Celsius, preferably, about 55 to 75 degrees Celsius for about 40 minutes, thereby the fibrous substrate 10 is bonded with the mixture to form a composition 15 and the composition 15 will not transfer to other materials.

In some embodiments, the mixture further comprises cholesterol. For instance, the mixture comprises 70 percent beeswax, 25 percent mineral oil, and 5 percent cholesterol with respect to total weight of the mixture.

Still in another embodiment, the fibrous substrate 10 may be dipped, soaked, tumbled, physically kneaded, or a combination thereof in the mixture 15 until the glove is uniformly colored.

In an alternative embodiment, the fibrous substrate 10 is thermally cured for about 90 minutes. It is anticipated that the thermal curing time may differ according to the quantity, weight or type of the fibrous substrate 10. The thermal curing process increases binding of the mixture to the article and prevents the mixture from transferring to another material when in use or shipped.

While the embodiments of the disclosure have been disclosed, certain modifications may be made by those skilled in the art to modify the disclosure without departing from the spirit of the disclosure.

1. A device for delivering topicals, comprising:
a fibrous substrate comprising at least a predetermined percentage of cotton;
wherein the fibrous substrate comprises pores;
a composition homogenously bonded to the pores of the fibrous substrate;
wherein the composition comprises a predetermined percentage of beeswax and a predetermined percentage of mineral oil; and wherein the predetermined percentage of beeswax is greater than the predetermined percentage of mineral oil.

2. The delivery system as described in claim 1, wherein the delivery system has an occlusion rate of less than 4000 g/m²·day.

3. The delivery system as described in claim 1, wherein the predetermined percentage of cotton is 50 percent with respect to total weight of the fibrous substrate.

4. The delivery system as described in claim 1, wherein the fibrous substrate further comprises one or more materials selected from the group of consisting of polyester, spandex, nylon, silver and antimicrobial mesh.

5. The delivery system as described in claim 1, wherein the predetermined percentage of beeswax is 65 to 75 percent with respect to total weight of the composition, and the predetermined percentage of mineral oil is 25 to 35 percent with respect to total weight of the composition.

6. The delivery system as described in claim 1, wherein the fibrous substrate further comprises a predetermined percentage of polyester, and the predetermined percentage of cotton is 50 to 100 percent with respect to total weight of the fibrous substrate and the predetermined percentage of polyester is less than 1 percent to to 50 percent with respect to total weight of the fibrous substrate.

7. The delivery system as described in claim 1, wherein the fibrous substrate is a patch, a bandage, or a garment.

8. The delivery system as described in claim 7, wherein the garment is a baby suit, a glove, a sock, or a sleeve.

9. A delivery system for delivering topicals, comprising:
a fibrous substrate comprising a predetermined percentage of cotton and a predetermined percentage of polyester;
wherein the fibrous substrate comprises pores;
wherein the predetermined percentage of cotton is equal to or greater than the predetermined percentage of polyester; and
a composition homogenously bonded to the pores of the fibrous substrate;
wherein the composition comprises a predetermined percentage of beeswax and a predetermined percentage of mineral oil.

10. The delivery system as described in claim 9, wherein the delivery system has an occlusion rate of less than 4000 g/m²·day.

11. The delivery system as described in claim 9, wherein the predetermined percentage of cotton is 50 to 100 percent and the predetermined percentage of polyester is less than 1 percent to 50 percent with respect to a total weight of the fibrous substrate.

12. The delivery system as described in claim 9, wherein the fibrous substrate further comprises one or more materials selected from the group of consisting of spandex, nylon, silver and antimicrobial mesh.

13. The delivery system as described in claim 9, wherein the predetermined percentage of beeswax is 65 to 75 percent with respect to a total weight of the composition, and the predetermined percentage of mineral oil is 25 to 35 percent with respect to the total weight of the composition.

14. The delivery system as described in claim 9, wherein the fibrous substrate is a patch, a bandage, or a garment.

15. A delivery system for delivering topicals, comprising:
a fibrous substrate comprising at least a predetermined percentage of cotton;
wherein the fibrous substrate comprises continuous pores;
a composition homogenously bonded to the pores of the fibrous substrate;
wherein the composition comprises a predetermined percentage of beeswax, a predetermined percentage of mineral oil, and a predetermined percentage of cholesterol; and wherein the predetermined percentage of beeswax is greater than the predetermined percentage of mineral oil and the predetermined percentage of cholesterol.

16. The delivery system as described in claim 15, wherein the delivery system has an occlusion rate of less than 4000 g/m²·day.

17. The delivery system as described in claim 15, wherein the predetermined percentage of cotton is 50 percent with respect to a total weight of the fibrous substrate.

18. The delivery system as described in claim 15, wherein the fibrous substrate further comprises one or more materials selected from the group consisting of polyester, spandex, nylon, silver and antimicrobial mesh.

19. The delivery system as described in claim 15, wherein the predetermined percentage of beeswax is 70 percent, the predetermined percentage of mineral oil is 25 percent, and the predetermined percentage of cholesterol is 5 percent with respect to a total weight of the composition.

20. The delivery system as described in claim 15, wherein the fibrous substrate is a patch, a bandage, or a garment.
